(19) United States
(12) Patent Application Publication
(10) Pub. No.: US 2025/0017928 A1
(43) Pub. Date: Jan. 16, 2025

(54) OPHTHALMIC PHARMACEUTICAL COMPOSITIONS CONTAINING A NITRIC OXIDE-RELEASING PHOSPHODIESTERASE 5 INHIBITOR AND METHOD FOR THE PREPARATION THEREOF

(71) Applicant: Nicox SA, Biot (FR)

(72) Inventors: Nicoletta ALMIRANTE, Milano (IT); Stefania BRAMBILLA, Merone (IT); Corinna GALLI, Milano (IT)

(21) Appl. No.: 18/714,300

(22) PCT Filed: Dec. 1, 2022
(86) PCT No.: PCT/EP2022/083986

§ 371 Date: May 29, 2024

(30) Foreign Application Priority Data
Dec. 2, 2021 (EP) 21211990.3

(51) Int. Cl. A61K 31/506; A61K 9/00; A61K 47/02; A61K 47/10; A61K 47/14; A61K 47/18; A61K 47/26

(52) U.S. Cl. CPC A61K 31/506; A61K 9/0048; A61K 47/02; A61K 47/10; A61K 47/14; A61K 47/183; A61K 47/186; A61K 47/26

(57) ABSTRACT

The present invention discloses aqueous ophthalmic formulations in the form of eye drops comprising [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate able to retain physical and chemical stability of the active ingredient over time.

The present invention relates to aqueous ophthalmic formulations in the form of eye drops comprising [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate capable of maintaining physical and chemical stability of the active substance in the dosage form and a method for the preparation of such ophthalmic formulations.

[(2S)-1-(4-{[(3-Chloro-4-methoxyphenyl)methyl]amino}-5-{[(pyrimidin-2-yl) methyl]carbamoyl}pyrimidin-2-yl)pyrrolidin-2-yl]methyl 6-(nitrooxy)hexanoate is a nitric oxide releasing phosphodiesterase 5 inhibitor (NO-PDE5 inhibitor), hereafter referred to as Compound (I).

Compound (I) is disclosed in WO 2020/030489 that relates to NO-releasing PDE5 inhibitors and their use for the treatment of ocular diseases associated with elevated intraocular pressure, such as ocular hypertension and glaucoma, and for treating retinopathies. WO 2020/030489 does not disclose specific pharmaceutical formulations comprising said NO-releasing PDE5 inhibitors.

One obstacle to the preparation of an ophthalmic formulation comprising Compound (I) in the commonly used aqueous ophthalmic vehicles is its rather low aqueous solubility (0.013 mg/ml) being it largely lipophilic (clog D_{pH 6.7}=5.76).

In an effort to enhance the solubility of Compound (I) in aqueous vehicle, ‘salt forms’ of Compound (I) were explored. However, the tested Compound (I) salts exhibited similar aqueous solubility as that of Compound (I) free base in the range of pH 6.0-7.0, which was the pH initially chosen for the ophthalmic formulation.

Additionally, making salts requires additional processing steps which increase costs of synthesis: furthermore, it decreases the overall reaction yield.

Therefore, there is a need for an aqueous formulation of Compound (I) containing therapeutically effective amounts of the active ingredient, is easily manufactured, has a long shelf life under normal handling conditions, and is well tolerated by the eye.

The present invention provides an ophthalmic composition in the form of aqueous solution comprising the following ingredients:
(i) [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy)hexanoate (Compound (I)) as the active ingredient,
(ii) a surfactant component consisting of a mixture of two non-ionic surfactants selected from the following: Macrogolglycerol ricinoleate (Kolliphor® EL), Macrogol stearate 40 (Myrj™ S40), and Polyoxyethylenesorbitan monooleate (Tween® 80),
(iii) a co-solvent selected from the class of polyols which also serves as an osmolality adjuster;
(iv) a buffer system to adjust the pH over a range of 4.5 to 7.5;
(v) water for injection or purified water;

The ophthalmic composition of the invention is in the form of eye drops for topical ocular administration.

The inventors have found that the combination of the surfactant component and the presence of a polyol are able to enhance the solubility of Compound (I) in aqueous vehicle and the resulting clear ophthalmic formulation is largely stable without insoluble deposit formation over time. Furthermore, when tested in animals it resulted well tolerated.

An embodiment of the invention provides an ophthalmic formulation comprising:
(i) 0.8% to 1.8% w/w of [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate or any specific value within said range;
(ii) a surfactant component selected from the following mixtures:
   4.0% to 7.5% w/w Macrogolglycerol ricinoleate and 1.0% to 6.0% w/w Macrogol stearate 40,
   4.0% to 7.5% w/w Macrogolglycerol ricinoleate and 1.0% to 7.0% w/w Polyoxyethylenesorbitan monooleate, or
   1.0% to 6.0% w/w Macrogol stearate 40 and 1.0% to 7.0% w/w Polyoxyethylenesorbitan monooleate;
(iii) 1.0% to 5.0% w/w of a co-solvent selected from the class of polyols;
(iv) a buffer system to adjust the pH in a range of 4.5 to 7.5;
(v) water for injection or purified water q.s. to 100% w/w,
and wherein the osmolality of the ophthalmic formulations is 240 to 400 mOsm/kg, preferably 240 to 380 mOsm/kg.

Another embodiment of the invention provides an ophthalmic formulation comprising:
(i) 1.0% to 1.8% w/w of [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate or any specific value within said range;
(ii) a surfactant component selected from the following mixtures;
   5.0% to 7.5% w/w Macrogolglycerol ricinoleate and 3.0% to 5.0% w/w Macrogol stearate 40,
   5.0% to 7.5% w/w of Macrogolglycerol ricinoleate and 2.0% to 5.0% w/w Polyoxyethylenesorbitan monooleate, or
   3.0% to 5.0% w/w Macrogol stearate 40 and 2.0% to 5.0% w/w Polyoxyethylenesorbitan monooleate;
(iii) 1.0% to 4.0% w/w Polyethylene glycol 400;
(iv) a buffer system to adjust the pH to 4.5 to 7.5;
(v) water for injection or purified water q.s. to 100% w/w;
and further comprising 0.01% to 0.10% w/w EDTA and 0.01% to 0.02% w/w benzalkonium chloride, and
wherein the osmolality of the ophthalmic formulations is 240 to 400 mOsm/kg, preferably 240 to 380 mOsm/kg.

An embodiment of the invention provides an ophthalmic formulation comprising:
(i) 1.0% to 1.5% w/w of [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy)hexanoate or any specific value within said range;
(ii) a surfactant component selected from the following mixtures:
   5.0% to 7.5% w/w Macrogolglycerol ricinoleate and 3.0% to 5.0% w/w Macrogol stearate 40,
   5.0% to 7.5% w/w Macrogolglycerol ricinoleate and 2.0% to 5.0% w/w Polyoxyethylenesorbitan monooleate, or
   3.0% to 5.0% w/w Macrogol stearate 40 and 2.0% to 5.0% w/w Polyoxyethylenesorbitan monooleate;
(iii) 1.0% to 4.0% w/w of a co-solvent selected from the class of polyols;
(iv) a buffer system to adjust the pH over a range of 4.5 to 7.5;
(v) water for injection or purified water q.s. to 100% w/w, and
wherein the osmolality of the ophthalmic formulations is 240 to 400 mOsm/kg, preferably 240 to 380 mOsm/kg.

In the formulations of the present invention the co-solvent of the class of polyols is preferably selected from mannitol, glycerol, sorbitol or polyethylene glycols: most preferably the co-solvent is Polyethylene glycol 400.

In the formulations of the present invention, the buffer system is preferably selected from the following: boric acid and disodium hydrogen phosphate heptahydrate, disodium hydrogen phosphate heptahydrate and sodium dihydrogen phosphate dihydrate, disodium hydrogen phosphate heptahydrate and citric acid, trisodium citrate dihydrate and citric acid monohydrate, trisodium citrate dihydrate and boric acid, boric acid. More preferably the buffer system is a mixture of boric acid and disodium hydrogen phosphate heptahydrate or citric acid monohydrate and trisodium citrate dehydrate or trisodium citrate dihydrate and boric acid or boric acid.

Optionally the ophthalmic composition further comprises other excipients such as a chelating agent and an antimicrobial preservative.

Preferably, the chelating agent is ethylenediaminetetraacetic acid (EDTA) that is present in the formulation in an amount from 0.01% to 0.2% w/w. Within the terms of the present invention, EDTA relates to ethylenediamine tetraacetic acid itself as well to its salts, namely e.g. disodium and/or potassium salts.

The antimicrobial preservative may be selected from quaternary ammonium compounds such as benzalkonium chloride (BAK), polyquaternium-1 (PQ-1), benzethonium chloride, potassium sorbate or sorbic acid or a mixture thereof, preferably the antimicrobial preservative is benzalkonium chloride.

Benzalkonium chloride is better described as: N-benzyl-N-(C₈-C₁₈alkyl)-N,N-dimethylammonium chloride. Preferably, the antimicrobial preservative is benzalkonium chloride. Benzalkonium chloride is present in the solution in an amount from 0.01% to 0.02% w/w.

Optionally hydrochloric acid and/or sodium hydroxide may be added to ophthalmic composition to adjust the pH of the final formulation to a pH over a range of 4.5 to 7.5.

Other optional excipients that may be included in the ophthalmic formulation are: tonicity adjusting agents such as glycerol, sorbitol, mannitol, dextrose, sodium or potassium chloride, viscosity enhancing agents such as methyl cellulose, hydroxypropyl methylcellulose, antioxidants or a mixture thereof.

A preferred embodiment of the invention provides an ophthalmic formulation consisting essentially of:
(i) 1.0% w/w [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate;
(ii) 5.0% w/w Macrogolglycerol ricinoleate and 3.0% w/w Macrogol stearate 40;
(iii) 2.8% w/w Polyethylene glycol 400;
(iv) 0.19% w/w boric acid, 0.51% w/w disodium hydrogen phosphate heptahydrate;
(v) water for injection or purified water q.s. to 100% w/w;
and further comprising 0.1% w/w disodium ethylenediaminetetraacetate dihydrate, 0.01% w/w benzalkonium chloride and HCl or NaOH to adjust the pH of the final solution to 6.5-6.9.

Another preferred embodiment of the invention provides an ophthalmic formulation consisting essentially of:
(i) 1.3% w/w [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate;
(ii) 7.5% w/w Macrogolglycerol ricinoleate and 5.0% w/w Macrogol stearate 40;
(iii) 2.8% w/w Polyethylene glycol 400;
(iv) 0.19% w/w boric acid, 0.51% w/w disodium hydrogen phosphate heptahydrate;
(v) water for injection or purified water q.s. to 100% w/w;
and further comprising 0.1% w/w disodium ethylenediaminetetraacetate dihydrate, 0.01% w/w benzalkonium chloride, and HCl or NaOH to adjust pH to 6.5-6.9.

Another preferred embodiment of the invention provides an ophthalmic formulation consisting essentially of:
(i) 1.4% w/w [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate;
(ii) 7.5% w/w Macrogolglycerol ricinoleate and 5.0% w/w Macrogol stearate 40;
(iii) 4.0% w/w Polyethylene glycol 400;
(iv) 0.19% w/w boric acid, 0.51% w/w disodium hydrogen phosphate heptahydrate;
(v) water for injection or purified water q.s. to 100% w/w;
and further comprising 0.1% w/w disodium ethylenediaminetetraacetate dihydrate, 0.01% w/w benzalkonium chloride, and HCl or NaOH to adjust pH to 6.5-6.9.

Another preferred embodiment of the invention provides an ophthalmic formulation consisting essentially of:
(i) 1.8% w/w [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate;
(ii) 7.5% w/w Macrogolglycerol ricinoleate and 5.0% w/w Polyoxyethylenesorbitan monooleate;
(iii) 1.0% w/w Polyethylene glycol 400;
(iv) 0.19% w/w boric acid, 0.51% w/w disodium hydrogen phosphate heptahydrate;
(v) water for injection or purified water q.s. to 100% w/w;
and further comprising 0.1% w/w disodium ethylenediaminetetraacetate dihydrate, 0.01% w/w benzalkonium chloride, and HCl or NaOH to adjust pH to 6.5-6.9.

Another preferred embodiment of the invention provides an ophthalmic formulation consisting essentially of:
(i) 1.7% w/w [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate;
(ii) 7.5% w/w Macrogolglycerol ricinoleate and 5.0% w/w Polyoxyethylenesorbitan monooleate;
(iii) 1.0% w/w Polyethylene glycol 400;
(iv) 0.19% w/w boric acid, 0.51% w/w disodium hydrogen phosphate heptahydrate;
(v) water for injection or purified water q.s. to 100% w/w;
and further comprising 0.1% w/w disodium ethylenediaminetetraacetate dihydrate, 0.01% w/w benzalkonium chloride.

Another preferred embodiment of the invention provides an ophthalmic formulation consisting essentially of:
(i) 1.7% w/w [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate;
(ii) 7.5% w/w Macrogolglycerol ricinoleate and 5.0% w/w Polyoxyethylenesorbitan monooleate;
(iii) 1.0% w/w Polyethylene glycol 400;
(iv) 0.11% w/w citric acid monohydrate and 0.73% w/w trisodium citrate dihydrate;
(v) water for injection or purified water q.s. to 100% w/w;
and further comprising 0.1% w/w disodium ethylenediaminetetraacetate dihydrate, 0.01% w/w benzalkonium chloride.

Another preferred embodiment of the invention provides an ophthalmic formulation consisting essentially of:
(i) 1.7% w/w [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate;
(ii) 7.5% w/w Macrogolglycerol ricinoleate and 5.0% w/w Polyoxyethylenesorbitan monooleate;
(iii) 1.0% w/w Polyethylene glycol 400;
(iv) 0.19% w/w boric acid, 0.003% w/w trisodium citrate dihydrate;
(v) water for injection or purified water q.s. to 100% w/w;
and further comprising 0.1% w/w disodium ethylenediaminetetraacetate dihydrate, 0.01% w/w benzalkonium chloride.

Another preferred embodiment of the invention provides an ophthalmic formulation consisting essentially of:
(i) 1.7% w/w [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate;
(ii) 7.5% w/w Macrogolglycerol ricinoleate and 5.0% w/w Polyoxyethylenesorbitan monooleate;
(iii) 1.0% w/w Polyethylene glycol 400;
(iv) 0.11% w/w citric acid monohydrate, 0.73% w/w trisodium citrate dihydrate;
(v) water for injection or purified water q.s. to 100% w/w;
and further comprising 0.1% w/w disodium ethylenediaminetetraacetate dihydrate, 0.01% w/w benzalkonium chloride.

Another preferred embodiment of the invention provides an ophthalmic formulation consisting essentially of:
(i) 1.7% w/w [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate;
(ii) 7.5% w/w Macrogolglycerol ricinoleate and 5.0% w/w Polyoxyethylenesorbitan monooleate;
(iii) 1.0% w/w Polyethylene glycol 400;
(iv) 0.11% w/w citric acid monohydrate, 0.73% w/w trisodium citrate dihydrate;
(v) water for injection or purified water q.s. to 100% w/w;
and further comprising 0.1% w/w disodium ethylenediaminetetraacetate dihydrate, 0.01% w/w benzalkonium chloride.

The percentages of the components of the ophthalmic formulations of the invention are expressed with respect to the total weight of the formulation.

Another embodiment of the present invention provides the use of the ophthalmic composition of the invention for treating glaucoma, ocular hypertension, pathological conditions associated with elevated intraocular pressure or retinopathies.

The topical ophthalmic formulations of the present invention can be prepared using a process that comprises the following steps:
1) adding under stirring into the preparation vessel:
   two of the following non-ionic surfactants: Macrogolglycerol ricinoleate (Kolliphor® EL), Macrogol stearate 40 (Myrj™ S40) and Polyoxyethylenesorbitan monooleate (Tween® 80),
   a co-solvent,
   at least 80% of the final weight of water for injection or purified water,
   optionally an antimicrobial preservative,
and stirring the mixture to obtain a solution;
2) adding a buffer system and stirring the mixture until complete solubilization, measuring the pH and optionally adjusting the pH between 4.5 to 7.5;
3) heating up the solution at 65° C., then adding [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate (Compound (I)) and stirring the mixture until dissolution of Compound (I);
4) cooling down the solution to room temperature (RT) under stirring;
5) optionally adding a chelating agent and stirring the mixture for complete solubilization;
6) optionally, adjusting the pH value to 4.5-7.5, preferably to pH 6.5-6.9;
7) adjusting the final weight of the solution with water for injection or purified water;
8) checking and adjusting the pH, if necessary, to 4.5-7.5, preferably to pH 6.5-6.9;
9) sterile filtering the bulk solution using a suitable filter with a nominal pore size of 0.2 μm or lower.

In particular, the topical ophthalmic formulations of the present invention can be prepared using a process that comprises the following steps:
1) adding under stirring into the preparation vessel:
   two of the following non-ionic surfactants: Macrogolglycerol ricinoleate (Kolliphor® EL), Macrogol stearate 40 (Myrj™ S40) and Polyoxyethylenesorbitan monooleate (Tween® 80),
   a co-solvent such as Polyethylene glycol 400 (Kollisolv® PEG E 400),
   80% to 90% of the final weight of water for injection or purified water,
   an antimicrobial preservative such as benzalkonium chloride,
and stirring the mixture to obtain a solution:
2) adding a buffer system such as boric acid (H₃BO₃) and disodium hydrogen phosphate heptahydrate (Na₂HPO₄·7H₂O) and stirring the mixture until complete solubilization, measuring the pH and optionally adjusting the pH with HCl or NaOH between 4.5 to 7.5;
3) heating up the solution at 65° C., then adding [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate (Compound (I)) and stirring the mixture until dissolution of Compound (I);
4) cooling down the solution to room temperature (RT) under stirring;
5) adding a chelating agent such as disodium ethylenediaminetetraacetate dihydrate (EDTA) and stirring the mixture for complete solubilization;
6) optionally, adjusting the pH value to 4.5-7.5, preferably 6.5-6.9 by HCI and/or NaOH;
7) adjusting the final weight of the solution with water for injection or purified water;
8) checking and adjusting the pH, if necessary, to 4.5-7.5, preferably 6.5-6.9 by adding NaOH and/or HCl;
9) sterile filtering the bulk solution using a suitable filter with a nominal pore size of 0.2 μm or lower.

### EXAMPLES

[(2S)-1-(4-{[(3-chloro-4-methoxyphenyl)methyl]amino}-5-{[(pyrimidin-2-yl)methyl] carbamoyl}pyrimidin-2-yl)pyrrolidin-2-yl]methyl 6-(nitrooxy)hexanoate (Compound (I)) was prepared according to the method of synthesis disclosed in WO 2020/030489. Macrogolglycerol ricinoleate (Kolliphor® EL), Macrogol stearate 40 (Myrj™ S40), Polyoxyethylenesorbitan monooleate (Tween® 80) and Polyethylene glycol 400 (Kollisolv® PEG E 400) are commercially available. The surfactants and co-solvents conform to the requirements of the European and US Pharmacopoeias.

The percentages of the components of the ophthalmic formulation are expressed with respect to the total weight of the formulation (% w/w).

Q.s. (quantum satis) means “Add as much of an ingredient as is needed to achieve the desired result”.

### Example 1

This example illustrates the preparation of an ophthalmic formulation of the invention containing 1.0% w/w of [(2S)-1-(4-{[(3-chloro-4-methoxyphenyl)methyl] amino}-5-{[(pyrimidin-2-yl)methyl]carbamoyl}pyrimidin-2-yl)pyrrolidin-2-yl]methyl 6-(nitrooxy)hexanoate (Compound (I)).

5.0 g of Macrogolglycerol ricinoleate (Kolliphor® EL), 3.0 g of Macrogol stearate 40 (Myrj™ S40), 2.8 g of Polyethylene glycol 400 (Kollisolv® PEG 400), 0.01 g of benzalkonium chloride, and 69.9 g of water for injection were weighted into the preparation vessel. The mixture was stirred using a magnetic stir-plate until all components were dissolved and the achieved solution appeared to be slightly yellow or colorless and transparent. 0.19 g of boric acid (H₃BO₃) and 0.51 g of disodium hydrogen phosphate heptahydrate (Na₂HPO₄ 7H₂O) were added and the mixture was stirred until complete dissolution. The system was then heated up to 65° C. before adding 1 g of Compound (I) and stirring for 30 minutes. The solution was then cooled down to room temperature (RT) under stirring. 0.10 g of disodium ethylenediaminetetraacetate dihydrate (EDTA) was added and further stirred to have a clear solution. The pH of the formulation was then measured and adjusted to 6.5-6.9 by adding an aqueous solution of HCl (0.5M). The remaining water for injection to reach the final weight of 100 g was added and the pH was measured again. The bulk formulation was then filtered under sterile conditions by means of suitable filter with a nominal pore size of 0.2 μm.

### Example 2

This example illustrates the preparation of an ophthalmic formulation of the invention containing 1.3% w/w of [(2S)-1-(4-{[(3-chloro-4-methoxyphenyl)methyl] amino}-5-{[(pyrimidin-2-yl)methyl]carbamoyl}pyrimidin-2-yl)pyrrolidin-2-yl]methyl 6-(nitrooxy) hexanoate (Compound (I)).

7.5 g of Macrogolglycerol ricinoleate (Kolliphor® EL), 5.0 g of Macrogol stearate 40 (Myrj™ S40), 2.8 g of Polyethylene glycol 400 (Kollisolv® PEG 400), 0.01 g of benzalkonium chloride, and 66.1 g of water for injection were weighted into the preparation vessel. The mixture was stirred using a magnetic stir-plate until all components were dissolved and the resulting solution appeared as slightly yellow or colorless and transparent. 0.19 g of boric acid (H₃BO₃) and 0.51 g of disodium hydrogen phosphate heptahydrate (Na₂HPO₄·7H₂O) were added and the mixture was stirred until complete dissolution of all ingredients. The system was then heated up to 65° C. before adding 1.3 g of Compound (I) followed by additional 30 minutes stirring. The solution was then cooled down to room temperature (RT) under stirring. 0.10 g of disodium ethylenediaminetetraacetate dihydrate was added and the solution was stirred until complete solubilization. The pH of the resulting formulation was measured and adjusted to 6.5-6.9 by adding an aqueous solution of HCl (0.5 M). The remaining water for injection to reach the final weight of 100 g was added the pH was measured again. The bulk formulation was finally filtered under sterile conditions by means of suitable filter with a nominal pore size of 0.2 μm.

### Example 3

This example illustrates the preparation of an ophthalmic formulation of the invention containing 1.4% w/w of [(2S)-1-(4-{[(3-chloro-4-methoxyphenyl)methyl] amino}-5-{[(pyrimidin-2-yl)methyl]carbamoyl}pyrimidin-2-yl)pyrrolidin-2-yl]methyl 6-(nitrooxy)hexanoate (Compound (I)).

7.5 g of Macrogolglycerol ricinoleate (Kolliphor® EL), 5.0 g of Macrogol stearate 40 (Myrj™ S40), 4.0 g of Polyethylene glycol 400 (Kollisolv® PEG 400), 0.01 g of benzalkonium chloride, and 65.0 g of water for injection were weighted into the preparation vessel. The mixture was stirred using a magnetic stir-plate until all components were dissolved and the achieved solution appeared to be slightly yellow or colorless and transparent. 0.19 g of boric acid (H₃BO₃) and 0.51 g of disodium hydrogen phosphate heptahydrate (Na₂HPO₄·7H₂O) were added and the mixture was stirred until complete dissolution. The system was then heated up to 65° C. before adding 1.4 g of Compound (I) and stirring for 30 minutes. The solution was then cooled down to room temperature (RT) under stirring. 0.10 g of disodium ethylenediaminetetraacetate dihydrate was added and the solution was stirred until complete solubilization. The pH of the achieved formulation was measured and adjusted to 6.5-6.9 by adding an aqueous solution of HCl (0.5 M). The remaining water for injection to reach the final weight of 100 g was added the pH was measured again. The bulk formulation was then filtered under sterile conditions by means of suitable filter with a nominal pore size of 0.2 μm.

### Example 4

This example illustrates the preparation of an ophthalmic formulation of the invention containing 1.8% w/w of [(2S)-1-(4-{[(3-chloro-4-methoxyphenyl)methyl] amino}-5-{[(pyrimidin-2-yl)methyl]carbamoyl}pyrimidin-2-yl)pyrrolidin-2-yl]methyl 6-(nitrooxy) hexanoate (Compound (I)).

7.5 g of Macrogolglycerol ricinoleate (Kolliphor® EL), 5.0 g of Polyoxyethylenesorbitan monooleate (Tween® 80), 1.0 g of Polyethylene glycol 400 (Kollisolv® PEG 400), 0.01 g of benzalkonium chloride, and 67.1 g of water for injection were weighted into the preparation vessel. The mixture was stirred using a magnetic stir-plate until all components were dissolved and the achieved solution appeared to be slightly yellow or colorless and transparent. 0.19 g of boric acid (H₃BO₃) and 0.51 g of disodium hydrogen phosphate heptahydrate (Na₂HPO₄·7H₂O) were added and the mixture was stirred until complete dissolution. The system was then heated up to 65° C. before adding 1.8 g of Compound (I) and stirring for 30 minutes. The solution was then cooled down to room temperature (RT) under stirring. 0.10 g of disodium ethylenediaminetetraacetate dihydrate was added and the solution was stirred until complete solubilization. The pH of the achieved formulation was measured and adjusted to 6.5-6.9 by adding an aqueous solution of HCl (0.5 M). The remaining water for injection to reach the final weight of 100 g was added and pH was measured again. The bulk formulation was then filtered under sterile conditions by means of suitable filter with a nominal pore size of 0.2 μm.

### Example 5

### Stability Studies of Formulation Described in Example 1

The ophthalmic formulation of Example 1 was stored at 25° C. for stability analysis. The samples were analyzed at different time points up to 37 weeks. The results are reported in Table 1.

The amount of Compound (I) was determined with an HPLC equipped with an UV detector and using a mixture of two mobile phases (A: ammonium acetate 10 mM adjusted to pH 4.55 by adding acetic acid 50%; B: methanol).

TABLE 1
Stability data of ophthalmic formulation of Example 1

| | Time (weeks) | | | | | | | | |
|---|---|---|---|---|---|---|---|---|---|
| | t0 | | | | | | | | |
| | (release) | t4 | t8 | t13 | t17 | t21 | t25 | t27 | t37 |
| pH | 6.6 | 6.6 | 6.5 | 6.6 | 6.5 | 6.4 | 6.6 | 6.5 | ND |
| Compound (I) | 96.6 | 96.3 | 96.1 | 95.7 | 95.3 | 95.5 | 95.1 | 94.8 | 94.2 |
| Purity | | | | | | | | | |
| (area %) | | | | | | | | | |
| Compound (I) | 1.1 | 1.1 | 1.2 | 1.1 | 1.1 | 1.1 | 1.1 | 1.1 | 1.2 |
| Quantification | | | | | | | | | |
| (% w/w) | | | | | | | | | |
| Osmolality | ND | ND | 322 | ND | 327 | ND | ND | ND | ND |
| (mOsm/kg) | | | | | | | | | |

| | |
|---|---|
| Macrosopic | At each time point the formulations appeared |
| appearance | colourless, free of visible particles |

### Example 6

### Stability Studies of Formulation Described in Example 2

The ophthalmic formulation of Example 2 was stored at 25° C. for stability analysis. The samples were analyzed at different time points up to 8 weeks. The results are reported in Table 2.

The amount of Compound (I) was determined with an HPLC equipped with an UV detector and using a mixture of two mobile phases (A: ammonium acetate 10 mM adjusted to pH 4.55 by adding acetic acid 50%; B: methanol).

TABLE 2
Stability data of formulation of Example 2

| Time (weeks) | t0 (Release) | t2 | t4 | t8 |
|---|---|---|---|---|
| pH | 6.7 | 6.6 | 6.8 | 6.5 |
| Compound (I) | 96.8 | 96.4 | 96.5 | 96.2 |
| Purity (area %) | | | | |
| Compound (I) | 1.4 | 1.4 | 1.3 | 1.3 |
| Quantification | | | | |
| (% w/w) | | | | |

| | |
|---|---|
| Macroscopic | At each time point the formulations appeared |
| appearance | slightly yellow, free of visible particles |

### Example 7

### Stability Studies of Formulation Described in Example 3

The ophthalmic formulation of Example 3 was stored at 25° C. for stability analysis. The samples were analyzed at different time points up to 8 weeks. The results are reported in Table 3.

The amount of Compound (I) was determined with an HPLC equipped with an UV detector and using a mixture of two mobile phases (A: ammonium acetate 10 mM adjusted to pH 4.55 by adding acetic acid 50%; B: methanol).

TABLE 3
Stability data of formulation of Example 3

| Time (weeks) | t0 (Release) | t2 | t4 | t8 |
|---|---|---|---|---|
| pH | 6.7 | 6.7 | 6.9 | 6.6 |
| Compound (I) | 96.7 | 96.4 | 96.4 | 96.2 |
| Purity (area %) | | | | |
| Compound (I) | 1.5 | 1.5 | 1.4 | 1.4 |
| Quantification | | | | |
| (% w/w) | | | | |

| | |
|---|---|
| Macroscopic | At each time point the formulations appeared |
| appearance | slightly yellow, free of visible particles |

### Example 8

### Tolerability Study

The ophthalmic formulation of Example 1 was studied in non-human primates as well as in New Zealand White rabbits. Specifically, the ophthalmic formulation of Example 1 or the correspondent aqueous vehicle (5.0% w/w Macrogolglycerol ricinoleate and 3.0% w/w Macrogol stearate 40, 2.8% w/w Polyethylene glycol 400, 0.19% w/w boric acid, 0.51% w/w disodium hydrogen phosphate heptahydrate, 0.1% w/w disodium ethylenediaminetetraacetate dihydrate, 0.01% w/w benzalkonium chloride, HCl and water for injection q.s. to 100% w/w, pH 6.5-6.9) were administered acutely in non-human primates and repeatedly twice daily for up to 4 weeks in New Zealand White rabbits. In all the testing groups the administration of the ophthalmic formulation as well as that of the respective vehicle did not result in appreciable signs of discomfort as determined by visual inspection.

### Example 9

This example illustrates the preparation of an ophthalmic formulation of the invention containing 1.7% w/w of [(2S)-1-(4-{[(3-chloro-4-methoxyphenyl)methyl] amino}-5-{[(pyrimidin-2-yl)methyl]carbamoyl}pyrimidin-2-yl)pyrrolidin-2-yl]methyl 6-(nitrooxy)hexanoate (Compound (I)).

Macrogolglycerol ricinoleate (Kolliphor® EL) 1.5 g, Polyoxyethylenesorbitan monooleate (Tween® 80) 1.0 g, Polyethylene glycol 400 (Kollisolv® PEG E 400) 200 mg, benzalkonium chloride 2 mg and water for injection 13.5 g were weighted into the preparation vessel. The mixture was stirred using a magnetic stir-plate until obtaining a slightly yellow or colorless and transparent solution. Boric acid (H₃BO₃) 38 mg was added and the mixture was stirred until complete dissolution. The mixture was heated up to 65° C., then Compound (I) 340 mg was added and the mixture was stirred for 30 minutes. The solution was cooled down to room temperature (RT) under stirring, disodium ethylenediaminetetraacetate dihydrate 20 mg was added and the mixture was stirred until complete solubilization. The pH of the formulation was 5.6. Water for injection was added to adjust the final weight to 20 g. The resulting mixture was a solution. The bulk solution was then filtered under sterile conditions by means of suitable filter with a nominal pore size of 0.2 μm.

### Example 10

This example illustrates the preparation of an ophthalmic formulation of the invention containing 1.7% w/w of [(2S)-1-(4-{[(3-chloro-4-methoxyphenyl)methyl] amino}-5-{[(pyrimidin-2-yl)methyl]carbamoyl}pyrimidin-2-yl)pyrrolidin-2-yl]methyl 6-(nitrooxy)hexanoate (Compound (I)).

Macrogolglycerol ricinoleate (Kolliphor® EL) 1.5 g, Polyoxyethylenesorbitan monooleate (Tween® 80) 1.0 g, Polyethylene glycol 400 (Kollisolv® PEG E 400) 200 mg, benzalkonium chloride 2 mg, water for injection 13.5 g were weighted into the preparation vessel. The mixture was stirred using a magnetic stir-plate until after a slightly yellow or colorless and transparent solution was obtained. Citric acid monohydrate (C₆H₈O₇·H₂O, 22 mg) and trisodium citrate dihydrate (C₆H₅Na₃O₇·2H₂O), 146 mg) were added and the mixture was stirred until after all ingredients were dissolved. The mixture was heated up to 65° C. and then Compound (I) 340 mg was added followed by 30 minutes continuous stirring. The solution was then cooled down to room temperature (RT) under stirring. Disodium ethylenediaminetetraacetate dihydrate 20 mg was added and the solution was stirred to obtain a clear solution. The pH of the formulation was 5.8. Water for injection was added to adjust the final weight to 20 g. The resulting mixture was a solution. The bulk solution obtained was finally filtered under sterile conditions by means of a suitable filter with a nominal pore size of 0.2 μm.

### Example 11

This example illustrates the preparation of an ophthalmic formulation of the invention containing 1.7% w/w of [(2S)-1-(4-{[(3-chloro-4-methoxyphenyl)methyl] amino}-5-{[(pyrimidin-2-yl)methyl]carbamoyl}pyrimidin-2-yl)pyrrolidin-2-yl]methyl 6-(nitrooxy)hexanoate (Compound (I)).

Macrogolglycerol ricinoleate (Kolliphor® EL) 1.5 g, Polyoxyethylenesorbitan monooleate (Tween® 80) 1.0 g, Polyethylene glycol 400 (Kollisolv® PEG E 400) 200 mg, benzalkonium chloride 2 mg and water for injection 13.5 g were weighted into the preparation vessel. The mixture was stirred using a magnetic stir-plate to obtain a slightly yellow or colorless and transparent solution. Boric acid (H₃BO₃). 38 mg and trisodium citrate dihydrate (C₆H₅Na₃I₇·2H₂O) 0.6 mg were added and the mixture was stirred until complete dissolution. The mixture was heated up to 65° C., Compound (I) 340 mg was added and the resulting mixture stirred for 30 additional minutes. The solution was cooled down to room temperature (RT) under stirring. Disodium ethylenediaminetetraacetate dihydrate 20 mg was added and the mixture stirred until all ingredients were completely dissolved. The pH was 5.6. Water for injection was added to adjust the final weight to 20 g. The resulting mixture was a solution. The resulting bulk solution was later filtered under sterile conditions by means of a suitable filter with a nominal pore size of 0.2 μm.

### Example 12

This example illustrates the preparation of an ophthalmic formulation of the invention containing 1.7% w/w of [(2S)-1-(4-{[(3-chloro-4-methoxyphenyl)methyl] amino}-5-{[(pyrimidin-2-yl)methyl]carbamoyl}pyrimidin-2-yl)pyrrolidin-2-yl]methyl 6-(nitrooxy)hexanoate (Compound (I)).

Macrogolglycerol ricinoleate (Kolliphor® EL) 750 mg, Polyoxyethylenesorbitan monooleate (Tween® 80) 500 mg, Polyethylene glycol 400 (Kollisolv® PEG E 400) 100 mg, benzalkonium chloride 1 mg, and water for injection 6.7 g were weighted into the preparation vessel. The mixture was stirred using a magnetic stir-plate until obtaining a slightly yellow or colorless and transparent solution. Citric acid monohydrate (C₆H₈O₇·H₂O) 11 mg and trisodium citrate dihydrate (C₆H₅Na₃O₇·2H₂O) 73 mg were added and the mixture was stirred until complete dissolution. The pH was adjusted to 5.0 by adding HCl 0.5 M dropwise. The system was then heated up to 65° C., Compound (I) 170 mg was added and the mixture was stirred for 30 minutes. The solution was cooled down to room temperature (RT) under stirring. Disodium ethylenediaminetetraacetate dehydrate 10 mg was added and the mixture was stirred until complete solubilization. Water for injection was added to adjust the final weight to 10 g. The resulting mixture was a solution. The bulk solution was then filtered under sterile conditions by means of suitable filter with a nominal pore size of 0.2 μm.

### Example 13

This example illustrates the preparation of an ophthalmic formulation of the invention containing 1.7% w/w of [(2S)-1-(4-{[(3-chloro-4-methoxyphenyl)methyl] amino}-5-{[(pyrimidin-2-yl)methyl]carbamoyl}pyrimidin-2-yl)pyrrolidin-2-yl]methyl 6-(nitrooxy)hexanoate (Compound (I)).

Macrogolglycerol ricinoleate (Kolliphor® EL) 750 mg, Polyoxyethylenesorbitan monooleate (Tween® 80) 500 mg, Polyethylene glycol 400 (Kollisolv® PEG E 400) 100 mg, benzalkonium chloride 1 mg and water for injection 6.7 g were weighted into the preparation vessel. The mixture was stirred using a magnetic stir-plate until obtaining a slightly yellow or colorless and transparent solution. Citric acid monohydrate (C₆H₈O₇·H₂O) 11 mg and trisodium citrate dihydrate (C₆H₅Na₃O₇·2H₂O) 73 mg were added and the mixture was stirred until complete dissolution. The pH was adjusted to 4.5 by adding HCl 0.5 M dropwise. The mixture was heated up to 65° C., Compound (I) 170 mg was added and the mixture was stirred for 30 minutes. The solution was cooled down to room temperature (RT) under stirring. Disodium ethylenediaminetetraacetate dihydrate 10 mg was added and the mixture was stirred until complete solubilization. Water for injection was added to adjust the final weight to 10 g. The resulting mixture was a solution. The bulk solution was then filtered under sterile conditions by means of suitable filter with a nominal pore size of 0.2 μm.

### Example 14

### Stability Studies of Formulation Described in Example 4

The ophthalmic formulation of Example 4 was stored at 25° C. for stability analysis. The samples were analyzed at t0 and up to 2 weeks. The results are reported in Table 4.

The amount of Compound (I) was determined with an HPLC equipped with an UV detector and using a mixture of two mobile phases (A: ammonium acetate 10 mM adjusted to pH 4.55 by adding acetic acid 50%; B: methanol).

TABLE 4
Stability data of formulation of Example 4

| Time (weeks) | t0 (Release) | t2 |
|---|---|---|
| pH | 6.8 | 6.9 |
| Compound (I) Purity | 96.1 | 96.1 |
| (area %) | | |
| Compound (I) | 1.8 | 1.8 |
| Quantification (% w/w) | | |

| | |
|---|---|
| Macroscopic appearance | At each time point the formulations |
| | appeared slightly yellow, free of |
| | visible particles |

### Example 15

### Stability Studies of Formulation Described in Example 9

The ophthalmic formulation of Example 9 was stored at 25° C. for stability analysis. The samples were analyzed at different time points up to 14 weeks. The results are reported in Table 5.

The amount of Compound (I) was determined with an HPLC equipped with an UV detector and using a mixture of two mobile phases (A: ammonium acetate 10 mM adjusted to pH 4.55 by adding acetic acid 50%; B: methanol).

TABLE 5
Stability data of formulation of Example 9

| Time (weeks) | t0 (Release) | t2 | t4 | t8 | t14 |
|---|---|---|---|---|---|
| pH | 5.6 | 5.5 | 5.4 | 5.5 | 5.5 |
| Compound (I) | 96.2 | 96.4 | 95.7 | 95.2 | 94.8 |
| Purity | | | | | |
| (area %) | | | | | |
| Compound (I) | 1.7 | 1.7 | 1.8 | 1.7 | 1.7 |
| Quantification | | | | | |
| (% w/w) | | | | | |

| | |
|---|---|
| Macroscopic | At each time point the formulations appeared |
| appearance | slightly yellow, free of visible particles |

### Example 16

### Stability Studies of Formulation Described in Example 10

The ophthalmic formulation of Example 10 was stored at 25° C. for stability analysis. The samples were analyzed at different time points up to 8 weeks. The results are reported in Table 6.

The amount of Compound (I) was determined with an HPLC equipped with an UV detector and using a mixture of two mobile phases (A: ammonium acetate 10 mM adjusted to pH 4.55 by adding acetic acid 50%; B: methanol).

TABLE 6
Stability data of formulation of Example 10

| Time (weeks) | t0 (Release) | t2 | t4 | t8 |
|---|---|---|---|---|
| pH | 5.8 | 5.9 | 5.8 | 5.7 |
| Compound (I) | 97.1 | 95.8 | 95.9 | 95.5 |
| Purity | | | | |
| (area %) | | | | |
| Compound (I) | 1.7 | 1.8 | 1.8 | 1.8 |
| Quantification | | | | |
| (% w/w) | | | | |

| | |
|---|---|
| Macroscopic | At each time point the formulations appeared |
| appearance | slightly yellow, free of visible particles |

### Example 17

### Stability Studies of Formulation Described in Example 11

The ophthalmic formulation of Example 11 was stored at 25° C. for stability analysis. The samples were analyzed at different time points up to 8 weeks. The results are reported in Table 7.

The amount of Compound (I) was determined with an HPLC equipped with an UV detector and using a mixture of two mobile phases (A: ammonium acetate 10 mM adjusted to pH 4.55 by adding acetic acid 50%; B: methanol).

TABLE 7
Stability data of formulation of Example 11

| Time (weeks) | t0 (Release) | t2 | t4 | t8 |
|---|---|---|---|---|
| pH | 5.6 | 5.6 | 5.5 | 5.5 |
| Compound (I) | 97.2 | 95.9 | 95.9 | 95.5 |
| Purity | | | | |
| (area %) | | | | |
| Compound (I) | 1.7 | 1.8 | 1.8 | 1.7 |
| Quantification | | | | |
| (% w/w) | | | | |

| | |
|---|---|
| Macroscopic | At each time point the formulation appeared as |
| appearance | slightly yellow solution, free of visible particles |

### Example 18

### Stability Studies of Formulation Described in Example 12

The ophthalmic formulation of Example 12 was stored at 25° C. for stability analysis. The samples were analyzed at different time points up to 15 weeks. The results are reported in Table 8.

The amount of Compound (I) was determined with an HPLC equipped with an UV detector and using a mixture of two mobile phases (A: ammonium acetate 10 mM adjusted to pH 4.55 by adding acetic acid 50%; B: methanol).

TABLE 8
Stability data of formulation of Example 12

| Time (weeks) | t0 (Release) | t2 | t4 | t8 | t15 |
|---|---|---|---|---|---|
| pH | 5.0 | 5.2 | 5.2 | 5.1 | 4.8 |
| Compound (I) | 96.3 | 95.6 | 95.0 | 94.3 | 93.4 |
| Purity | | | | | |
| (area %) | | | | | |
| Compound (I) | 1.8 | 1.7 | 1.7 | 1.7 | 1.6 |
| Quantification | | | | | |
| (% w/w) | | | | | |

| | |
|---|---|
| Macroscopic | At each time point the formulations appeared |
| appearance | slightly yellow, free of visible particles |

### Example 19

### Stability Studies of Formulation Described in Example 13

The ophthalmic formulation of Example 13 was stored at 25° C. for stability analysis. The samples were analyzed at different time points up to 15 weeks. The results are reported in Table 9.

The amount of Compound (I) was determined with an HPLC equipped with an UV detector and using a mixture of two mobile phases (A: ammonium acetate 10 mM adjusted to pH 4.55 by adding acetic acid 50%; B: methanol).

TABLE 9
Stability data of formulation of Example 13

| Time (weeks) | t0 (Release) | t2 | t4 | t8 | t15 |
|---|---|---|---|---|---|
| pH | 4.5 | 4.6 | 4.6 | 4.5 | 4.3 |
| Compound (I) | 96.2 | 95.3 | 94.1 | 92.5 | 90.4 |
| Purity | | | | | |
| (area %) | | | | | |
| Compound (I) | 1.8 | 1.7 | 1.7 | 1.6 | 1.5 |
| Quantification | | | | | |
| (% w/w) | | | | | |

| | |
|---|---|
| Macroscopic | At each time point the formulations appeared |
| appearance | slightly yellow, free of visible particles |

The stability data reported in Tables 1 to 9 show that the formulations of the invention are chemically and physically stable at room temperature conditions and therefore no refrigerated storage condition is required thereby promoting an easily drug supply chain and patient compliance with the drug storage recommendations.

### Comparative Examples

The following examples disclose the preparation of ophthalmic formulations containing [(2S)-1-(4-{[(3-chloro-4-methoxyphenyl)methyl]amino}-5-{[(pyrimidin-2yl)methyl] carbamoyl}pyrimidin-2-yl)pyrrolidin-2-yl]methyl 6-(nitrooxy)hexanoate (Compound (I)), the vehicles of the ophthalmic formulations of the comparative examples 20 to 24 contain the same “surfactant component” and co-solvent of the formulations of the invention, but the amount of the “surfactant component” and the co-solvent are less than the amounts of the invention. The ophthalmic formulations of comparative examples 25 and 26 contain only a surfactant at a concentration within the range of the invention while the concentration of the co-solvent is higher than the concentration of the co-solvent of the formulation of the invention. The formulations of the comparative examples were prepared by adding 1% w/w of Compound (I) (examples 20-23 and 25) or 1.5% w/w of Compound (I) (examples 24 and 25), at the end of the preparation the macroscopic appearance of the formulations and the amount of Compound (I) dissolved in the vehicle of the formulations were evaluated.

### Example 20

Macrogolglycerol ricinoleate (Kolliphor® EL) 350 g, Macrogol stearate 40 (Myrj™ S40) 50 mg, Polyethylene glycol 400 (Kollisolv® PEG E 400) 50 mg, benzalkonium chloride 1 mg and water for injection 9.3 g were weighted into the preparation vessel. The mixture was stirred using a magnetic stir-plate until obtaining a slightly yellow or colorless solution. Boric acid (H₃BO₃) 19 mg and disodium hydrogen phosphate heptahydrate (Na₂HPO₄·7H₂O) 51 mg were added and the mixture was stirred until complete dissolution. The solution was heated up to 65° C., then Compound (I) 100 mg was added and the mixture was stirred for 30 minutes. The mixture was cooled to room temperature (RT) under stirring. Disodium ethylenediaminetetraacetate dihydrate (EDTA) 10 mg was added and the mixture was stirred for 10 minutes. Water for injection was added to adjust the final weight to 10 g. The pH was 7.6.

The resulting mixture was a suspension. The suspension was centrifuged and the amount of Compound (I) in the supernatant solution (vehicle) was quantified by HPLC against a calibration curve. The formulation was stored at RT, 31 weeks after the preparation a sample of the suspension was centrifuged and the amount of Compound (I) in the supernatant solution was quantified by HPLC against a calibration curve. The results of the two analyses are reported in the table below (Table 10).

TABLE 10
Stability data of formulation of Example 20

| Time (weeks) | t0 (Release) | t31 |
|---|---|---|
| Macroscopic | White, milky | White, milky |
| appearance | suspension | suspension |
| pH | 7.6 | 7.4 |
| Compound (I) | 0.6 | 0.5 |
| Quantification | | |
| (% w/w) | | |
| Compound (I) | 96.7 | 93.7 |
| Purity | | |
| (area %) | | |

### Example 21

Macrogolglycerol ricinoleate (Kolliphor® EL) 350 mg, Polyoxyethylenesorbitan monooleate (Tween® 80) 50 mg, Polyethylene glycol 400 (Kollisolv® PEG E 400) 50 mg, benzalkonium chloride 1 mg and water for injection 9.3 g were weighted into the preparation vessel. The mixture was stirred using a magnetic stir-plate until obtaining a slightly yellow or colorless solution. Boric acid (H₃BO₃) 19 mg and disodium hydrogen phosphate heptahydrate (Na₂HPO₄·7H₂O) 51 mg were added and the mixture was stirred until complete dissolution. The solution was heated up to 65° C., then Compound (I) 100 mg was added and the mixture was stirred for 30 minutes. The mixture was cooled down to room temperature (RT) under stirring. Disodium ethylenediaminetetraacetate dihydrate (EDTA) 10 mg was added and the mixture was stirred for 10 minutes. Water for injection was added to adjust the final weight to 10 g. The pH was 7.6.

The resulting mixture was a suspension. The suspension was centrifuged and the amount of Compound (I) in the supernatant solution (vehicle) was quantified by HPLC against a calibration curve. The formulation was stored at RT, 30 weeks after the preparation a sample of the suspension was centrifuged and the amount of Compound (I) in the supernatant solution was quantified by HPLC against a calibration curve. The results of the two analyses are reported in the table below (Table 11).

TABLE 11
Stability data of formulation of Example 21

| Time (weeks) | t0 (Release) | t30 |
|---|---|---|
| Macroscopic | White, milky | White, milky |
| appearance | suspension | suspension |
| pH | 7.6 | 7.4 |
| Compound (I) | 0.6 | 0.5 |
| Quantification | | |
| (% w/w) | | |
| Compound (I) | 96.8 | 93.7 |
| Purity | | |
| (area %) | | |

### Example 22

Macrogolglycerol ricinoleate (Kolliphor® EL) 350 mg, Macrogol stearate 40 (Myrj™ S40) 50 mg, Polyethylene glycol 400 (Kollisolv® PEG E 400) 50 mg, benzalkonium chloride 1 mg and water 9 g for injection were weighted into the preparation vessel. The mixture was stirred using a magnetic stir-plate until obtaining a slightly yellow or colorless solution. Boric acid (H₃BO₃) 19 mg and disodium hydrogen phosphate heptahydrate (Na₂HPO₄·7H₂O) 51 mg were added and the mixture was stirred until complete dissolution. The pH was adjusted to 3.1-4.2 by adding HCl 0.5 M. The solution was heated up to 65° C., then Compound (I) 100 mg was added and the mixture was stirred for 30 minutes. The mixture was cooled down to room temperature (RT) under stirring. Disodium ethylenediaminetetraacetate dihydrate (EDTA) 10 mg was added and the mixture was stirred for 1 hour. Water for injection was added to adjust the final weight to 10 g. The pH was 4.2.

The resulting mixture was a suspension. The suspension was centrifuged and the amount of Compound (I) in the supernatant solution (vehicle) was quantified by HPLC against a calibration curve. The formulation was stored at RT, 27 weeks after the preparation, a sample of the suspension was centrifuged and the amount of Compound (I) in the supernatant solution was quantified by HPLC against a calibration curve. The results of the two analyses are reported in the table below (Table 12).

TABLE 12
Stability data of formulation of Example 22

| Time (weeks) | t0 (Release) | t27 |
|---|---|---|
| Macroscopic | White, milky | White, milky |
| appearance | suspension | suspension |
| pH | 4.2 | 4.2 |
| Compound (I) | 0.8 | 0.5 |
| Quantification | | |
| (% w/w) | | |
| Compound (I) | 96.8 | 86.8 |
| Purity | | |
| (area %) | | |

### Example 23

Macrogolglycerol ricinoleate (Kolliphor® EL) 350 mg, Polyoxyethylenesorbitan monooleate (Tween® 80) 50 mg, of Polyethylene glycol 400 (Kollisolv® PEG E 400) 50 mg, benzalkonium chloride 1 mg and water for injection 9 g were weighted into the preparation vessel. The mixture was stirred using a magnetic stir-plate until obtaining a slightly yellow or colorless solution. Boric acid (H₃BO₃) 19 mg and disodium hydrogen phosphate heptahydrate (Na₂HPO₄·7H₂O) 51 mg were added; the mixture was stirred until complete dissolution. The pH was adjusted to 3.1-4.2 by adding dropwise HCl 0.5 M. The solution was heated up to 65° C., then Compound (I) 100 mg was added and the mixture was stirred for 30 minutes. The mixture was cooled down to room temperature (RT) under stirring. Disodium ethylenediaminetetraacetate dihydrate (EDTA) 10 mg was added and the mixture was stirred for 1 hour. Water for injection was added to adjust the final weight to 10 g. The pH was 4.2.

The resulting mixture was a suspension. The suspension was centrifuged and the amount of Compound (I) in the supernatant solution (vehicle) was quantified by HPLC against a calibration curve. The formulation was stored at RT, 27 weeks after the preparation, a sample of the suspension was centrifuged and the amount of Compound (I) in the supernatant solution was quantified by HPLC against a calibration curve. The results of the two analyses are reported in the table below (Table 13).

TABLE 13
Stability data of formulation of Example 23

| Time (weeks) | t0 (Release) | t27 |
|---|---|---|
| Macroscopic | White suspension | White suspension |
| appearance | | |
| pH | 4.2 | 4.3 |
| Compound (I) | 0.8 | 0.6 |
| Quantification | | |
| (% w/w) | | |
| Compound (I) | 96.9 | 88.8 |
| Purity | | |
| (area %) | | |

### Example 24

Macrogolglycerol ricinoleate (Kolliphor® EL) 350 mg, Macrogol stearate 40 (Myrj™ S40) 50 mg, Polyethylene glycol 400 (Kollisolv® PEG E 400) 550 mg, benzalkonium chloride 1 mg and water for injection 8 g were weighted into the preparation vessel. The mixture was stirred using a magnetic stir-plate until obtaining a slightly yellow or colorless solution. Boric acid (H₃BO₃) 19 mg and disodium hydrogen phosphate heptahydrate (Na₂HPO₄·7H₂O) 51 mg were added; the mixture was stirred until complete dissolution. The solution was heated up to 65° C., then Compound (I) 150 mg was added and the mixture was and stirred for 30 minutes. The mixture was cooled down to room temperature (RT) under stirring and disodium ethylenediaminetetraacetate dihydrate (EDTA) 10 mg was added and the mixture was stirred for 20 minutes. Water for injection was added to adjust the final weight to 10 g. The pH was 7.6.

The resulting mixture was a suspension. The suspension was centrifuged and the amount of Compound (I) in the supernatant solution (vehicle) was quantified by HPLC against a calibration curve. The formulation was stored at RT, 2 weeks after the preparation, an aliquot was withdrawn, centrifuged and the amount of Compound (I) in the supernatant solution was quantified by HPLC against a calibration curve. The results of the two independent analyses are reported in the table below (Table 14).

TABLE 14
Stability data of formulation of Example 24

| Time (weeks) | t0 (Release) | t2 |
|---|---|---|
| Macroscopic | White, milky | White, milky |
| appearance | suspension | suspension |
| pH | 7.6 | 7.4 |
| Compound (I) | 0.6 | 0.5 |
| Quantification | | |
| (% w/w) | | |
| Compound (I) | 99.1 | 98.9 |
| Purity | | |
| (area %) | | |

### Example 25

Macrogol stearate 40 (Myrj™ S40) 650 mg, Polyethylene glycol 400 (Kollisolv® PEG E 400) 50 mg, benzalkonium chloride 1 mg and water for injection 9.1 g were weighted into the preparation vessel. The mixture was stirred using a magnetic stir-plate until a slightly yellow or colorless solution was obtained. Boric acid (H₃BO₃) 19 mg and disodium hydrogen phosphate heptahydrate (Na₂HPO₄·7H₂O) 51 mg were added; the mixture was stirred until complete dissolution. The solution was heated up to 65° C., then Compound (I) 100 mg was added and the mixture was stirred for additional 30 minutes. The mixture was cooled down to room temperature (RT) under stirring and disodium ethylenediaminetetraacetate dihydrate (EDTA) 10 mg was added while stirring for 10 more minutes. Water for injection was added to adjust the final weight to 10 g. The pH was 7.6.

The resulting mixture was a suspension. The suspension was centrifuged and the amount of Compound (I) in the supernatant solution (vehicle) was quantified by HPLC against a calibration curve. The formulation was stored at RT, 30 weeks after the preparation an aliquot was withdrawn, centrifuged and the amount of Compound (I) quantified by HPLC against a calibration curve. The results of two independent analyses are reported in the table below (Table 15).

TABLE 15
Stability data of formulation of Example 25

| Time (weeks) | t0 (Release) | t30 |
|---|---|---|
| Macroscopic | White, milky | Clear solution |
| appearance | suspension | without visible |
| | | particles |
| pH | 7.6 | 7.4 |
| Compound (I) | 0.4 | 0.3 |
| Quantification | | |
| (% w/w) | | |
| Compound (I) | 96.7 | 92.0 |
| Purity | | |
| (area %) | | |

### Example 26

Macrogol stearate 40 (Myrj™ S40) 650 mg, Polyethylene glycol 400 (Kollisolv® PEG E 400) 550 mg, benzalkonium chloride 1 mg and water for injection 8.5 g were weighted into the preparation vessel. The mixture was stirred using a magnetic stir-plate until obtaining a slightly yellow or colorless solution. Boric acid (H₃BO₃) 19 mg and disodium hydrogen phosphate heptahydrate (Na₂HPO₄·7H₂O) 51 mg were added; the mixture was stirred until complete dissolution. The solution was heated up to 65° C., then Compound (I) 150 mg was added and the mixture was and stirred for 30 minutes. The mixture was cooled down to room temperature (RT) under stirring. Disodium ethylenediaminetetraacetate dihydrate (EDTA) 10 mg was added and the mixture was stirred for 20 minutes. Water for injection was added to adjust the final weight to 10 g. The pH was 7.6.

The resulting mixture was a suspension. The suspension was centrifuged and the amount of Compound (I) in the supernatant solution (vehicle) was quantified by HPLC against a calibration curve. The formulation was stored at RT, 2 weeks after the preparation, a sample of the suspension was centrifuged and the amount of Compound (I) in the supernatant solution was quantified by HPLC against a calibration curve. The results of the two analyses are reported in the table below (Table 16).

TABLE 16
Stability data of formulation of Example 26

| Time (weeks) | t0 (Release) | t2 |
|---|---|---|
| Macroscopic | White, milky | Clear solution |
| appearance | suspension | without visible |
| | | particles |
| pH | 7.6 | 7.4 |
| Compound (I) | 0.6 | 0.6 |
| Quantification | | |
| (% w/w) | | |
| Compound (I) | 99.2 | 98.9 |
| Purity | | |
| (area %) | | |

### Comments

The formulations of the comparative examples contain the same surfactant components and co-solvent of those of the formulations of the invention but at lower concentrations (see examples 20 to 24). Alternatively, other formulations contain the surfactant and co-solvent at higher concentration than that in the formulation of the invention (examples 25 and 26).

All the formulations of the comparative examples were suspensions.

The results of the analyses of the formulations of the comparative examples showed that the concentrations of Compound (I) in the supernatant solution of the formulations of Examples 20, 21, 24 to 26 were below 0.8%. At lower pH (pH 4.2) the concentration of Compound (I) increased up to 0.8%, however, the pH had an impact on the degradation of the active principle, namely after 27 weeks of storage a decrease of about 8 to 10% purity was observed (see Examples 22 and 23).

A summary of the stability analyses of the comparative examples 20 to 26 and Example 1, that refers to an ophthalmic formulation of the invention containing 1% w/w of Compound (I) according to the invention, is reported in Table 17.

TABLE 17

| | | | | Kollisolv ® | | | | | |
|---|---|---|---|---|---|---|---|---|---|
| | Kolliphor ® | Myrj ™ | Tween ® | PEG E | | | | Compound | |
| | EL | S40 | 80 | 400 | | | Macroscopic | (I) | Purity |
| Example | (% w/w) | (% w/w) | (% w/w) | (% w/w) | pH | Time | appearance | (% w/w) | (area %) |
| 1 | 5.0 | 3.0 | 0 | 2.8 | 6.6 | t0 | Solution | 1.1 | 96.6 |
| 1 | 5.0 | 3.0 | 0 | 2.8 | ND | 37 | Solution | 1.2 | 94.2 |
| | | | | | | weeks | | | |
| 20 | 3.5 | 0.5 | 0 | 0.5 | 7.6 | t0 | White | 0.6 | 96.7 |
| | | | | | | | suspension | | |
| 20 | 3.5 | 0.5 | 0 | 0.5 | 7.4 | 31 | White | 0.5 | 93.7 |
| | | | | | | weeks | suspension | | |
| 21 | 3.5 | 0 | 0.5 | 0.5 | 7.6 | t0 | White | 0.6 | 96.8 |
| | | | | | | | suspension | | |
| 21 | 3.5 | 0 | 0.5 | 0.5 | 7.4 | 30 | White | 0.5 | 93.7 |
| | | | | | | weeks | suspension | | |
| 22 | 3.5 | 0.5 | 0 | 0.5 | 4.2 | t0 | White | 0.8 | 96.8 |
| | | | | | | | suspension | | |
| 22 | 3.5 | 0.5 | 0 | 0.5 | 4.2 | 27 | White | 0.5 | 86.8 |
| | | | | | | weeks | suspension | | |
| 23 | 3.5 | 0 | 0.5 | 0.5 | 4.2 | t0 | White | 0.8 | 96.9 |
| | | | | | | | suspension | | |
| 23 | 3.5 | 0 | 0.5 | 0.5 | 4.3 | 27 | White | 0.6 | 88.8 |
| | | | | | | weeks | suspension | | |
| 24 | 3.5 | 0.5 | 0 | 5.5 | 7.6 | t0 | White | 0.6 | 99.1 |
| | | | | | | | suspension | | |
| 24 | 3.5 | 0.5 | 0 | 5.5 | 7.4 | 2 | White | 0.5 | 98.9 |
| | | | | | | weeks | suspension | | |
| 25 | 0 | 6.5 | 0 | 0.5 | 7.6 | t0 | White | 0.4 | 96.7 |
| | | | | | | | suspension | | |
| 25 | 0 | 6.5 | 0 | 0.5 | 7.4 | 30 | White | 0.3 | 92.0 |
| | | | | | | weeks | suspension | | |
| 26 | 0 | 6.5 | 0 | 5.5 | 7.6 | t0 | White | 0.6 | 99.2 |
| | | | | | | | suspension | | |
| 26 | 0 | 6.5 | 0 | 5.5 | 7.4 | 2 | Clear | 0.6 | 98.9 |
| | | | | | | weeks | solution | | |

1. An ophthalmic formulation comprising:
(i) 0.8% to 1.8% w/w of [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy)hexanoate or any specific value within said range;
(ii) a surfactant component selected from the following mixtures:
4.0% to 7.5% w/w Macrogolglycerol ricinoleate and 1.0% to 6.0% w/w Macrogol stearate 40,
4.0% to 7.5% w/w Macrogolglycerol ricinoleate and 1.0% to 7.0% w/w Polyoxyethylenesorbitan monooleate, or
1.0% to 6.0% w/w Macrogol stearate 40 and 1.0% to 7.0% w/w Polyoxyethylenesorbitan monooleate;
(iii) 1.0% to 5.0% w/w of a co-solvent selected from the class of polyols;
(iv) a buffer system to adjust the pH in a range of 4.5 to 7.5;
(v) water for injection or purified water q.s. to 100% w/w,
and wherein the osmolality of the ophthalmic formulations is 240 to 400 mOsm/kg, preferably 240 to 380 mOsm/kg.

2. The ophthalmic formulation according to claim 1 comprising:
(i) 1.0% to 1.8% w/w of [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate or any specific value within said range;
(ii) a surfactant component selected from the following mixtures:
5.0% to 7.5% w/w Macrogolglycerol ricinoleate and 3.0% to 5.0% w/w Macrogol stearate 40,
5.0% to 7.5% w/w of Macrogolglycerol ricinoleate and 2.0% to 5.0% w/w Polyoxyethylenesorbitan monooleate, or
3.0% to 5.0% w/w Macrogol stearate 40 and 2.0% to 5.0% w/w Polyoxyethylenesorbitan monooleate;
(iii) 1.0% to 4.0% w/w Polyethylene glycol 400;
(iv) a buffer system to adjust the pH to 4.5 to 7.5;
(v) water for injection or purified water q.s. to 100% w/w;
and further comprising 0.01% to 0.10% w/w EDTA and 0.01% to 0.02% w/w benzalkonium chloride.

3. The ophthalmic formulation according to claim 1 comprising:
(i) 1.0% to 1.5% w/w of [(2S)-1-(4-{[(3-chloro-4-methoxyphenyl)methyl]amino}-5-{[(pyrimidin-2-yl)methyl]carbamoyl}-pyrimidin-2-yl)pyrrolidin-2-yl]methyl 6-(nitrooxy)hexanoate or any specific value within said range;
(ii) a surfactant component selected from the following mixtures:
5.0% to 7.5% w/w Macrogolglycerol ricinoleate and 3.0% to 5.0% w/w Macrogol stearate 40,
5.0% to 7.5% w/w Macrogolglycerol ricinoleate and 2.0% to 5.0% w/w Polyoxyethylenesorbitan monooleate, or
3.0% to 5.0% w/w Macrogol stearate 40 and 2.0% to 5.0% w/w Polyoxyethylenesorbitan monooleate;
(iii) 1.0% to 4.0% w/w of a co-solvent selected from the class of polyols;
(iv) a buffer system to adjust the pH over a range of 4.5 to 7.5;
(v) water for injection or purified water q.s. to 100% w/w.

4. The ophthalmic formulation according to claim 1 wherein the co-solvent is selected from mannitol, glycerol, sorbitol or polyethylene glycols.

5. The ophthalmic formulation according to claim 1 wherein the co-solvent is Polyethylene glycol 400.

6. The ophthalmic formulation according to claim 1 wherein the buffer system is selected from the following: boric acid and disodium hydrogen phosphate heptahydrate, disodium hydrogen phosphate heptahydrate and sodium dihydrogen phosphate dihydrate, disodium hydrogen phosphate heptahydrate and citric acid, trisodium citrate dihydrate and citric acid monohydrate, trisodium citrate dihydrate and boric acid, boric acid.

7. The ophthalmic formulation according to claim 1 wherein the buffer system is selected from boric acid and disodium hydrogen phosphate heptahydrate or citric acid monohydrate and trisodium citrate dehydrate or trisodium citrate dihydrate and boric acid or boric acid.

8. The ophthalmic formulation according to claim 1 further comprises a chelating agent.

9. The ophthalmic formulation according to claim 8 wherein the chelating agent is 0.01% to 0.2% w/w ethylenediaminetetraacetic acid or its salts.

10. The ophthalmic formulation according to claim 1 further comprising an antimicrobial preservative selected from benzalkonium chloride, polyquaternium-1, benzethonium chloride, potassium sorbate or sorbic acid or a mixture thereof.

11. The ophthalmic formulation according to claim 10 wherein the antimicrobial preservative is benzalkonium chloride.

12. The ophthalmic formulation according to claim 1 further comprising one or more tonicity adjusting agents selected from: glycerol, sorbitol, mannitol, dextrose, sodium or potassium chloride.

13. The ophthalmic formulation according to claim 1 further comprising a viscosity enhancing agent selected from methyl cellulose, hydroxypropyl methylcellulose, antioxidants or a mixture thereof.

14. The ophthalmic formulation according to claim 1 consisting essentially of:
(i) 1.0% w/w [(2S)-1-(4-{[(3-chloro-4-methoxyphenyl)methyl]amino}-5-{[(pyrimidin-2-yl)methyl] carbamoyl}pyrimidin-2-yl)pyrrolidin-2-yl]methyl 6-(nitrooxy)hexanoate;
(ii) 5.0% w/w Macrogolglycerol ricinoleate and 3.0% w/w Macrogol stearate 40;
(iii) 2.8% w/w Polyethylene glycol 400;
(iv) 0.19% w/w boric acid, 0.51% w/w disodium hydrogen phosphate heptahydrate;
(v) water for injection q.s. to 100% w/w;
and further comprising 0.1% w/w disodium ethylenediaminetetraacetate dihydrate, 0.01% w/w benzalkonium chloride and HCl or NaOH to adjust the pH to 6.5-6.9.

15. The ophthalmic formulation according to claim 1 consisting essentially of:
(i) 1.3% w/w [(2S)-1-(4-{[(3-chloro-4-methoxyphenyl)methyl]amino}-5-{[(pyrimidin-2-yl)methyl] carbamoyl}pyrimidin-2-yl)pyrrolidin-2-yl]methyl 6-(nitrooxy)hexanoate;
(ii) 7.5% w/w Macrogolglycerol ricinoleate and 5.0% w/w Macrogol stearate 40;
(iii) 2.8% w/w Polyethylene glycol 400;
(iv) 0.19% w/w boric acid, 0.51% w/w disodium hydrogen phosphate heptahydrate;
(v) water for injection q.s. to 100% w/w;
and further comprising 0.1% w/w disodium ethylenediaminetetraacetate dihydrate, 0.01% w/w benzalkonium chloride, and HCl or NaOH to adjust pH to 6.5-6.9.

16. The opthalmic formulation according to claim 1 consisting essentially of:
(i) 1.4% w/w [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy)hexanoate;
(ii) 7.5% w/w Macrogolglycerol ricinoleate and 5.0% w/w Macrogol stearate 40;
(iii) 4.0% w/w Polyethylene glycol 400;
(iv) 0.19% w/w boric acid, 0.51% w/w disodium hydrogen phosphate heptahydrate;
(v) water for injection q.s. to 100% w/w;
and further comprising 0.1% w/w disodium ethylenediaminetetraacetate dihydrate, 0.01% w/w benzalkonium chloride, and HCl to adjust pH to 6.5-6.9.

17. The ophthalmic formulation according to claim 1 consisting essentially of:
(i) 1.8% w/w [(2S)-1-(4-{[(3-chloro-4-methoxyphenyl)methyl]amino}-5-{[(pyrimidin-2-yl)methyl] carbamoyl}pyrimidin-2-yl)pyrrolidin-2-yl]methyl 6-(nitrooxy)hexanoate;
(ii) 7.5% w/w Macrogolglycerol ricinoleate and 5.0% w/w Polyoxyethylenesorbitan monooleate;
(iii) 1.0% w/w Polyethylene glycol 400;
(iv) 0.19% w/w boric acid, 0.51% w/w disodium hydrogen phosphate heptahydrate;
(v) water for injection q.s. to 100% w/w;
and further comprising 0.1% w/w disodium ethylenediaminetetraacetate dihydrate, 0.01% w/w benzalkonium chloride, and HCl or NaOH to adjust pH to 6.5-6.9.

18. The opthalmic formulation according to claim 1 consisting essentially of:
(i) 1.7% w/w [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate;
(ii) 7.5% w/w Macrogolglycerol ricinoleate and 5.0% w/w Polyoxyethylenesorbitan monooleate;
(iii) 1.0% w/w Polyethylene glycol 400;
(iv) 0.19% w/w boric acid, 0.51% w/w disodium hydrogen phosphate heptahydrate;
(v) water for injection or purified water q.s. to 100% w/w;
and further comprising 0.1% w/w disodium ethylenediaminetetraacetate dihydrate, 0.01% w/w benzalkonium chloride.

19. The ophthalmic formulation according to claim 1 consisting essentially of:
(i) 1.7% w/w [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate;
(ii) 7.5% w/w Macrogolglycerol ricinoleate and 5.0% w/w Polyoxyethylenesorbitan monooleate;
(iii) 1.0% w/w Polyethylene glycol 400;
(iv) 0.11% w/w citric acid monohydrate and 0.73% w/w trisodium citrate dihydrate;
(v) water for injection or purified water q.s. 100% w/w;
and further comprising 0.1% w/w disodium ethylenediaminetetraacetate dihydrate, 0.01% w/w benzalkonium chloride.

20. The ophthalmic formulation according to claim 1 consisting of essentially of:
(i) 1.7% w/w [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate;
(ii) 7.5% w/w Macrogolglycerol ricinoleate and 5.0% w/w Polyoxyethylenesorbitan monooleate;
(iii) 1.0% w/w Polyethylene glycol 400;
(iv) 0.19% w/w boric acid, 0.003% w/w trisodium citrate dihydrate;
(v) water for injection or purified water q.s. to 100% w/w;
and further comprising 0.1% w/w disodium ethylenediaminetetraacetate dihydrate, 0.01% w/w benzalkonium chloride.

21. The ophthalmic formulation according to claim 1 consisting essentially of:
(i) 1.7% w/w [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate;
(ii) 7.5% w/w Macrogolglycerol ricinoleate and 5.0% w/w Polyoxyethylenesorbitan monooleate;
(iii) 1.0% w/w Polyethylene glycol 400;
(iv) 0.11% w/w citric acid monohydrate, 0.73% w/w trisodium citrate dihydrate;
(v) water for injection or purified water q.s. to 100% w/w;
and further comprising 0.1% w/w disodium ethylenediaminetetraacetate dihydrate, 0.01% w/w benzalkonium chloride.

22. The ophthalmic formulation according to claim 1 consisting essentially of:
(i) 1.7% w/w [(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 6-(nitrooxy) hexanoate;
(ii) 7.5% w/w Macrogolglycerol ricinoleate and 5.0% w/w Polyoxyethylenesorbitan monooleate;
(iii) 1.0% w/w Polyethylene glycol 400;
(iv) 0.11% w/w citric acid monohydrate, 0.73% w/w trisodium citrate dihydrate;
(v) water for injection or purified water q.s. to 100% w/w;
and further comprising 0.1% w/w disodium ethylenediaminetetraacetate dihydrate, 0.01% w/w benzalkonium chloride.

23. The ophthalmic formulation according to claim 1 for use for treating glaucoma, ocular hypertension, pathological conditions associated with elevated intraocular pressure or retinopathies.
